(19) United States
(12) Patent Application Publication
(10) Pub. No.: US 2026/0263314 A1
(43) Pub. Date: Sep. 10, 2026

(54) HANDHELD MULTI-MODAL PHYSIOTHERAPY APPARATUS

(71) Applicant: Shenzhen Kaiyan Medical Equipment Co., Ltd., Shenzhen (CN)

(72) Inventors: Alain Dijkstra, Amstelveen (NL); Hu Chunlei, Shenzhen (CN); Peng Anle, Shenzhen (CN)

(73) Assignee: Shenzhen Kaiyan Medical Equipment Co., Ltd., Shenzhen (CN)

(21) Appl. No.: 19/558,543

(22) Filed: Mar. 6, 2026

(30) Foreign Application Priority Data
Mar. 7, 2025 (CN) 2025102659736

(51) Int. Cl. A61H 15/02; A61H 15/00; A61N 1/36; A61N 5/06

(52) U.S. Cl. CPC A61H 15/02; A61H 15/0092; A61H 2015/0042; A61H 2201/0157; A61N 1/36014; A61N 5/0616; A61N 2005/0651

(57) ABSTRACT

A therapeutic apparatus integrates rolling mechanical massage, electrical stimulation and phototherapy in a single device. A light-transmissive spherical treatment element carries patterned conductive regions that function as electrodes while optical windows allow passage of LED light. The ball is rotatably mounted on an electrically conductive support that provides continuous power and control despite rotation. A phototherapy unit assembly and control electronics are positioned within the housing to synchronize and modulate electrical and phototherapy. Contact and skin-quality sensors selectively activate only those electrodes and LEDs contacting skin, and the control system supports waveform modulation and periodic polarity reversal to reduce electrochemical effects and skin irritation. Floating mounts and a flexible housing permit conformal contact with facial and body contours. The design supports single or multiple spheres and multiple form factors for versatile topical therapy.

### FIELD OF INVENTION

The present invention relates to therapeutic skin treatment devices, and more particularly to a rolling ball therapeutic device combining electrical stimulation therapy and light therapy through a transparent ball.

### BACKGROUND

Therapeutic devices for skin treatment have evolved to incorporate various treatment modalities, including massage, electrical stimulation, and light therapy. Massage therapy devices may use rolling elements, such as spherical structures, to provide mechanical stimulation to the skin during use. Such rolling elements may conform to different body contours and may reduce friction during movement across skin surfaces compared to stationary treatment heads.

Electrical stimulation therapies, including microcurrent therapy, electrical muscle stimulation, galvanic therapy, and transcutaneous electrical nerve stimulation, have been applied to skin treatment applications. These therapies may involve the delivery of electrical current to skin tissue through electrodes positioned in contact with the skin surface. The electrodes may be incorporated into handheld devices that permit users to apply electrical stimulation to targeted treatment areas.

Light therapy devices have been developed to deliver electromagnetic radiation at therapeutic wavelengths to skin tissue. Light-emitting diodes may be configured to emit visible light or infrared radiation for phototherapy applications. The light sources may be positioned within a housing and oriented to direct light toward the skin tissue during treatment.

Some therapeutic devices have attempted to combine multiple treatment modalities within a single apparatus. For example, devices have incorporated spherical structures with stimulation elements such as LEDs, heating elements, and electrodes disposed on the outer surfaces. Other devices have utilized spherical bodies with grooves for plasma discharge and near-infrared irradiation. Still other devices have integrated bioelectricity, heating, vibration, and LED light functions into massage brush configurations with separate electrode assemblies.

However, existing multi-modality devices have certain limitations. Devices utilizing opaque or semi-opaque spherical materials may permit some light transmission but may not provide optimal optical clarity for targeted phototherapy delivery through specific regions of the sphere. Devices with grooves or channels for light emission may have a limited light-transmission area and may not permit simultaneous delivery of electrical stimulation and phototherapy through the same rolling contact surface. Devices with separate electrode assemblies, such as brush heads, may not provide the smooth rolling contact desirable for massage applications.

Electrical connections to rotating treatment elements present design considerations for therapeutic devices. The rotation of a treatment rolling ball during use may complicate electrical coupling between stationary circuit components and electrodes disposed on the rotating ball surface. Existing approaches may utilize wireless power transfer through induction coils, which adds complexity and may not provide continuous power during rapid rotation. Other approaches may utilize fixed electrode configurations that do not rotate with the treatment element.

Control systems for therapeutic devices may regulate the operation of treatment elements during use. Some devices control treatment parameters based on the user's skin type or pulse patterns. However, existing devices may not selectively activate only those treatment elements that are in contact with skin during use, potentially resulting in energy waste and off-target exposure. Additionally, devices utilizing continuous unidirectional DC stimulation may cause localized skin irritation due to electrochemical effects at the electrode-skin interface.

There remains a need for a therapeutic device that integrates rolling mechanical massage, electrical stimulation, and phototherapy through a single transparent spherical treatment element having patterned conductive traces that function as electrodes while permitting light transmission through optical windows defined between the conductive traces. Such a device would benefit from reliable electrical coupling to the rotating treatment element, selective activation of treatment elements based on skin contact, and polarity reversal or biphasic waveforms to reduce electrode polarization and skin irritation.

### OBJECTS OF THE INVENTION

Some of the objectives of the invention are as follows:

An object of the present invention is to provide a rolling ball therapeutic device configured to deliver phototherapy, electrical stimulation therapy, and massage therapy, either independently or simultaneously.

Another object of the present invention is to provide a therapeutic device that delivers simultaneous or sequential electrotherapy and phototherapy through a rotatable, light-transmissive sphere.

Another object of the present invention is to incorporate patterned conductive traces on a transparent sphere so that electrical stimulation can be applied while permitting light transmission.

Another object of the present invention is to enable reliable electrical coupling to a rotating treatment element without wire entanglement.

Another object of the present invention is to provide a therapeutic device with multiple spheres or sphere arrays to increase treatment coverage and uniformity.

Still another object of the present invention is to provide floating mounts and flexible housings so that the device conforms to varied skin contours.

Another object of the present invention is to provide a therapeutic device with an ergonomic housing and handle designs for precise user control.

Another object of the present invention is to offer a scalable architecture usable in handheld, jar-cap, mat, or massager product forms.

### SUMMARY OF THE INVENTION

According to a first aspect of the invention, a therapeutic device is provided. The therapeutic device comprises: a housing having a cavity; a rolling ball rotatably mounted in the cavity, wherein the rolling ball comprises a transparent material and has a conductive region and a light transmission region, the conductive region having at least one electrical stimulation element disposed thereon; a phototherapy unit positioned external to the rolling ball and within the cavity, the phototherapy unit configured to emit light through the light transmission region of the rolling ball; and a circuit board disposed within the cavity and electrically connected to the at least one electrical stimulation element and the phototherapy unit.

In one embodiment of the invention, the transparent material comprises at least one of glass, quartz, ceramic, or plastic.

In one embodiment of the invention, at least one electrical stimulation element comprises a patterned conductive trace disposed on a surface of the rolling ball.

In one embodiment of the invention, the patterned conductive trace comprises a metal coating applied to the surface of the rolling ball.

In one embodiment of the invention, at least one electrical stimulation element is configured to provide at least one of EMS therapy, microcurrent therapy, galvanic therapy, or TENS therapy.

In one embodiment of the invention, the phototherapy unit comprises at least one LED light source.

In one embodiment of the invention, the therapeutic device further comprises a conductive shaft disposed within the cavity, wherein the rolling ball is rotatably mounted on the conductive shaft, and wherein at least one electrical stimulation element is electrically connected to the circuit board through the conductive shaft.

In one embodiment of the invention, the therapeutic device further comprises a control circuit configured to selectively activate only those of at least one electrical stimulation element and the phototherapy unit that are in contact with skin during use.

In one embodiment of the invention, the control circuit is further configured to periodically reverse the polarity of electrical stimulation provided by at least one electrical stimulation element.

In one embodiment of the invention, at least two rolling balls are rotatably mounted in the cavity.

According to a second aspect of the invention, a therapeutic device is provided. The therapeutic device comprises: a transparent rolling ball having a plurality of light emitting diodes integrated therein; a plurality of patterned conductive traces disposed on an outer surface of the rolling ball, the patterned conductive traces configured to provide electrical stimulation therapy to skin while permitting light from the plurality of light emitting diodes to pass through optical windows defined between the patterned conductive traces; and a housing supporting the rolling ball, wherein the rolling ball is configured to roll along skin while simultaneously applying electrical stimulation and light therapy.

In one embodiment of the invention, the patterned conductive traces comprise a metal coating electroplated onto the outer surface of the rolling ball.

In one embodiment of the invention, the patterned conductive traces comprise raised electrode islands disposed on the outer surface of the rolling ball.

In one embodiment of the invention, the therapeutic device further comprises a control circuit configured to selectively activate only those of the plurality of light-emitting diodes and the patterned conductive traces that are in contact with skin during use.

In one embodiment of the invention, the control circuit is further configured to periodically reverse the polarity of electrical stimulation provided by the patterned conductive traces.

In one embodiment of the invention, the housing is configured as at least one of a handheld device, a jar cap, a mat, or a massager.

According to a third aspect of the invention, a method of providing therapeutic treatment to the skin is provided. The method comprises: rolling a ball along the skin, the ball comprising a transparent material, having at least one electrical stimulation element disposed on a surface thereof; delivering electrical stimulation therapy to the skin through the at least one electrical stimulation element while the ball rolls along the skin; and simultaneously delivering light therapy to the skin through a light transmission area of the transparent ball.

In one embodiment of the invention, at least one electrical stimulation element comprises a patterned conductive trace formed by electroplating a metal coating onto the surface of the transparent rolling ball.

In one embodiment of the invention, the method further comprises: selectively activating only those of the at least one electrical stimulation element that are in contact with the skin during use.

In one embodiment of the invention, the method further comprises: periodically reversing the polarity of the electrical stimulation.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term "several" means more than one, unless otherwise specified.

In the context of the specification, the term "phototherapy unit" refers to any device configured to emit therapeutic light for skin treatment, pain relief, or wellness applications.

In the context of the specification, the term “stimulation element” refers broadly to any component, module, or structure configured to apply a therapeutic or cosmetic stimulus to a user’s skin or tissue. Stimulation elements may include, but are not limited to, a phototherapy element, a microcurrent element, a Peltier element, a vibrational element, a thermal element, an ultrasonic wave therapy element, a magnetotherapy element, electrical stimulation elements, a galvanic element, a Tens element, an RF element, a pulsed electromagnetic field (PEMF) element or a combination thereof. In the context of the specification, the term “stimulation element” refers broadly to any component, module, or structure configured to apply a therapeutic or cosmetic stimulus to a user’s skin or tissue. The term “first stimulation element”, “second stimulation element”, and “third stimulation element” are for descriptive purpose and each term includes, but are not limited to, a phototherapy element, a microcurrent element, a Peltier element, a vibrational element, a thermal element, an ultrasonic wave therapy element, a magnetotherapy element, electrical stimulation elements, galvanic element, Tens element, RF element, a pulsed electromagnetic field (PEMF) element or a combination thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user’s skin. Such elements may include paired electrodes, conductive surfaces, or pads connected to a circuit board for generating microcurrent, EMS, galvanic current, or equivalent electrical therapy.

In the context of the specification, the term “housing” is intended to cover any casing, enclosure, or structural body that contains or supports components of the device. The housing may include a handle portion, head, or other segments, and may be made from polymeric, metallic, composite, or other suitable materials.

In the context of the specification, the term “control interface” refers to any input or output mechanism enabling a user to operate the device. The control interface may include physical buttons, capacitive touch sensors, sliders, switches, or graphical displays, and may further include wireless control via a mobile application.

In the context of the specification, the term “control or circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term "LED module" refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620–750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

In an embodiment, the phototherapy unit emits multiple therapeutic wavelengths adapted for skin care and dermatological treatment. Red light (approximately 630–660 nm) penetrates deeply into the skin to stimulate blood circulation, enhance collagen production, and promote skin regeneration and repair. Blue light (around 415–470 nm) exhibits antibacterial properties and is effective in treating acne, minimizing breakouts, and reducing inflammation. Green light (approximately 520–540 nm) helps reduce hyperpigmentation, even skin tone, and soothe sensitive or irritated skin. Yellow light (around 580–600 nm) improves cellular oxygen exchange, enhances lymphatic circulation, and supports detoxification and skin revitalization. Near-infrared light (800–850 nm) penetrates deeper tissue layers to accelerate healing, reduce pain, and alleviate inflammation, thereby supporting overall skin recovery and rejuvenation.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a therapeutic device with a rolling ball, in accordance with an embodiment of the present invention.

FIG. 2 shows an exploded view of the therapeutic device of FIG. 1, in accordance with an embodiment of the present invention.

FIG. 3 illustrates a configuration of the rolling ball of the therapeutic device, showing electrical stimulation elements, in accordance with an embodiment of the present invention.

FIG. 4 shows a perspective view of an alternate configuration of a therapeutic device having two rolling balls with patterned conductive traces, in accordance with an embodiment of the present invention.

FIG. 5 illustrates an exploded view of the therapeutic device shown in FIG. 4, in accordance with an embodiment of the present invention.

FIG. 6 shows another configuration of a therapeutic device integrated with a plurality of rolling balls and a plurality of massage spheres, in accordance with an embodiment of the present invention.

FIG. 7 illustrates an exploded view of a middle shell, the rolling ball and a conductive shaft of the therapeutic device given in FIG. 4, in accordance with an embodiment of the present invention.

### DETAILED DESCRIPTION OF THE INVENTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention provide a therapeutic device that integrates a rolling ball or massage sphere, electrical stimulation, and phototherapy within a single handheld assembly. The therapeutic device comprises a rolling ball rotatably mounted within a housing. The housing includes an opening through which a portion of the rolling ball protrudes to permit contact with a user’s skin during operation. The rolling ball is formed from a light-transmissive material and includes patterned conductive regions disposed on an outer surface thereof. The configuration enables simultaneous delivery of electrical stimulation through the conductive regions and phototherapy through light-transmissive regions of the rolling ball as the rolling ball rolls across the skin.

The housing defines an internal cavity configured to rotatably support the rolling ball. The internal cavity accommodates one or more phototherapy units or lamp board assemblies, a control circuit board, and a power source such as a battery. The housing can be configured in various form factors, including but not limited to a handheld unit, a jar-cap assembly, a mat structure, or a massager device. The housing can be formed from suitable polymeric materials, composites, or equivalent structural materials. In certain embodiments, a front portion of the housing is at least partially light-transmissive to facilitate emission of light toward the skin, while a rear portion functions as a light shield. The housing can comprise multiple shell components joined by snap-fit, threaded, magnetic, or other detachable engagement mechanisms to facilitate assembly and serviceability.

In certain embodiments, the rolling ball is formed from an optically clear or translucent material. An outer surface of the rolling ball incorporates patterned conductive traces or raised electrode islands arranged to define intervening light-transmissive regions. The conductive regions can be formed by metallization, conductive ink deposition, embedded conductive members, electroplating, vapour deposition, wire insertion, or equivalent fabrication techniques. The light-transmissive regions remain substantially free of opaque conductive material to permit efficient transmission of light emitted by internal phototherapy sources to the skin.

In certain embodiments, the rolling ball is rotatably supported by a conductive mounting shaft or similar support structure that additionally serves as an electrical pathway between the patterned conductive regions on the rolling ball and the control circuit. Electrical continuity during rotation may be maintained using compliant ring contacts, conductive pins or needles, conductive springs, slip ring assemblies, brush contacts, or other rotational contact interfaces configured to accommodate movement without wire entanglement. The mounting shaft may be retained within the housing by slots, clamping shells, floating retainers, bearings, or equivalent structures that permit free rotation of the rolling ball while maintaining structural stability.

In certain embodiments, one or more phototherapy units or lamp board assemblies are positioned within the housing opposite corresponding light-transmissive regions of the rolling ball. The lamp boards may be semicircular, segmented, circumferentially distributed, or replicated for multiple rolling balls such that light sources are aligned with the light-transmissive regions. In alternative implementations, one or more light-emitting elements may be embedded within the interior of the rolling ball itself, with patterned conductive regions disposed on the exterior surface to permit simultaneous internal light emission and external electrical stimulation.

In certain embodiments, a spring-mounted, resilient, or floating support structures permit vertical movement of each rolling ball, and associated massage sphere assembly, relative to the housing. Such movement enables the device to conform to facial or body contours during use. The housing can be formed from a flexible or partially flexible material to allow relative movement between multiple balls or massage sphere arrays under bending, thereby improving conformity to convex and concave surfaces. The device can further include an ergonomic handle, such as a C-shaped grip, and may incorporate soft over-molded surfaces to enhance user comfort and control during operation.

In an embodiment, the control circuit board and the power source supply electrical energy and execute predefined treatment protocols. The control electronics are configured to regulate stimulation waveforms, light intensity, timing, and sequencing of therapeutic outputs. One or more contact or proximity sensors, including capacitive sensors, pressure sensors, impedance sensors, or equivalent detection elements, may be provided to determine which portions of the rolling ball are in contact with the skin. Based on sensed contact information, the control system can selectively activate only those electrodes and light-emitting elements corresponding to skin-contacting regions, thereby conserving power and limiting unintended exposure. Grouping circuits, multiplexing arrangements, and switching matrices may be implemented to enable per-segment addressing of the patterned conductive regions and associated light sources.

In an embodiment, the control system is configured to generate a plurality of electrical stimulation waveforms, including but not limited to biphasic pulses, microcurrent protocols, and electrical muscle stimulation (EMS) bursts. The control system can periodically reverse polarity or apply charge-balanced biphasic waveforms to reduce electrochemical accumulation at the electrode-skin interface and to mitigate skin irritation. Polarity reversal and biphasic operation can further stabilize electrode-skin impedance and provide a more uniform perceived sensation. Safety features may include contact verification, current limiting, voltage regulation, and adaptive adjustment of intensity and treatment duration based on sensed skin parameters.

In certain embodiments, a detection probe or skin quality sensor is configured to measure one or more skin parameters, including impedance, moisture level, hydration state, temperature, or surface characteristics. Based on the measured parameters, the control system can adjust applied voltage and current levels, light intensity, waveform characteristics, and treatment duration. Such adaptive control may tailor therapy to individual skin conditions, thereby improving therapeutic efficacy while reducing the risk of overexposure or excessive stimulation.

In operation, the device is moved across the skin such that the rolling ball rolls along the treatment surface, causing the patterned conductive regions to sequentially contact the skin. Light-emitting elements aligned with corresponding light-transmissive regions of the rolling ball simultaneously irradiate the treatment area. The control system coordinates electrical and optical outputs by activating only those elements corresponding to regions in contact with the skin, modulating output levels in real time based on sensor feedback, and executing programmed waveform patterns, including polarity reversal where applicable.

The therapeutic device supports a range of implementation variants, including configurations having a single rolling ball, multiple rolling balls, massage spheres, or combinations thereof. In certain embodiments, rolling ball can incorporate internal light sources. Alternative form factors may include jar-cap assemblies, mat structures, or other configurations adapted for localized or larger-area applications. Variations can further include different electrode geometries, conductive materials, and electrical coupling strategies. The floating mount structures, flexible housing configurations, and sensing mechanisms may be optimized for specific anatomical regions such as the face, neck, or limbs. The spherical geometry promotes consistent surface contact, rolling action reduces sliding friction, and the arrangement of patterned conductive regions and light-transmissive regions enables concurrent electrotherapy and phototherapy. Selective activation and sensor-based control can enhance power efficiency and operational safety.

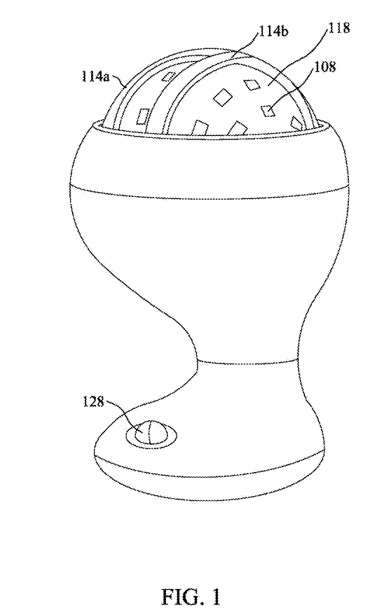

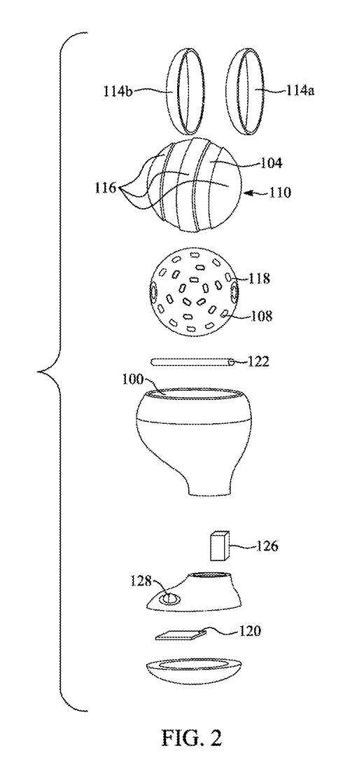

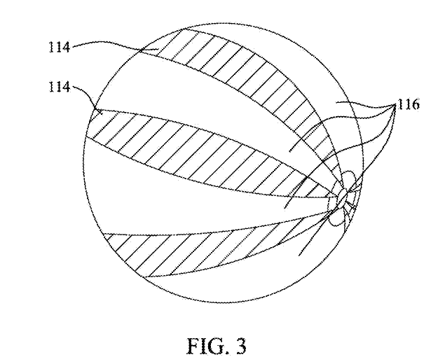

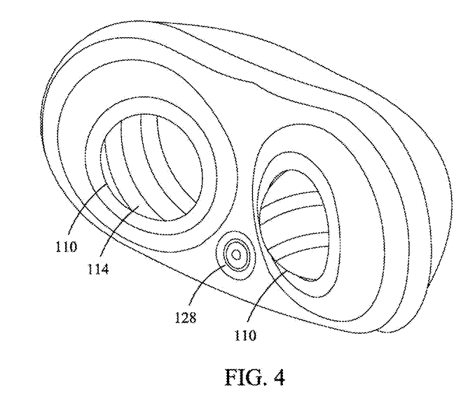

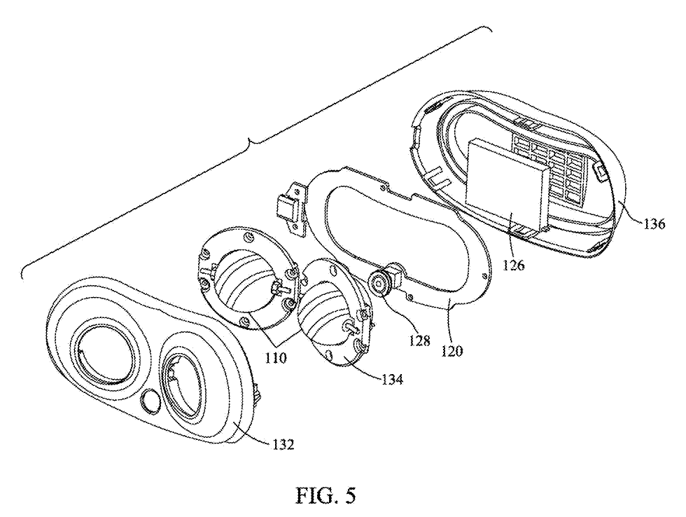

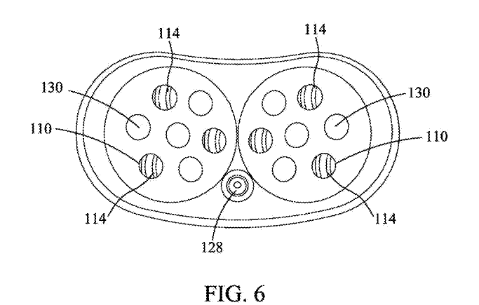

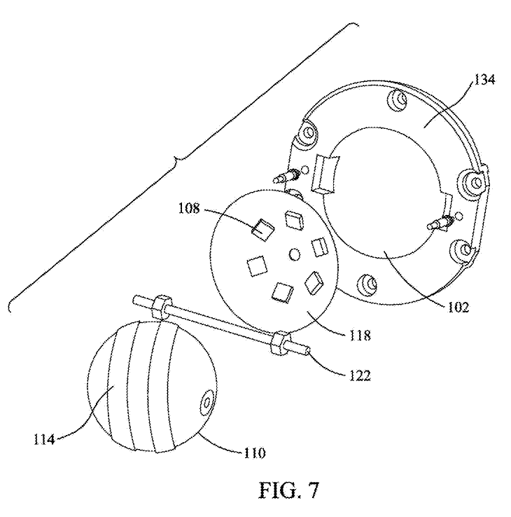

Embodiments of the present invention will now be described with reference to FIGS. 1 to 7.

Referring to FIG. 1, a therapeutic device is illustrated that integrates multiple treatment modalities within a single handheld apparatus. The therapeutic device combines electrical stimulation therapy and phototherapy delivered through a rolling ball mechanism. The therapeutic device is configured to provide simultaneous or sequential delivery of electrical stimulation and light therapy to skin tissue during use.

The therapeutic device comprises a housing 100 and a rolling ball 110 mounted within the housing 100 on a conductive shaft 122. The housing 100 includes an opening 102 through which a portion of the rolling ball 110 protrudes to contact the skin during therapeutic application. The rolling ball 110 is preferably fabricated from a transparent or translucent material and includes at least one conductive region disposed on an outer surface thereof, the conductive region comprising an electrical stimulation element 106 (also referred to as conductive strips or patterned conductive traces). The rolling ball 110 further defines at least one light-transmissive region 116. The housing 100 may be formed from suitable materials such as polyurethane (PU), thermoplastic polyurethane (TPU), or other appropriate polymeric materials.

The housing 100 further accommodates a phototherapy unit 118, which may include an array of light-emitting diodes (LEDs) 108 and associated optical components. The phototherapy unit 118 is positioned to emit therapeutic wavelengths through the light-transmissive region 116 of the rolling ball 110. A circuit board 120 is mounted within the housing 100 and is electrically coupled to the phototherapy unit 118 and to the electrical stimulation element 106 via the conductive shaft 122 or other electrical pathways. The circuit board 120 is configured to sequence and synchronize the operation of the electrical stimulation element 106 and the phototherapy unit 118, and to process signals from one or more sensors to ensure safe contact detection and proper timing of therapy delivery.

In an embodiment, the housing 100 defines an internal cavity configured to contain therapeutic components. The internal cavity accommodates the circuit board 120, a power source or a battery 126, one or more LEDs 108, and associated electrical connections. The internal cavity is dimensioned to permit rotatable mounting of the rolling ball 110 while allowing exposure of a portion of the rolling ball 110 through the opening 102 for skin contact during use.

In certain embodiments, the housing 100 comprises a front shell 132 and a rear shell 136 that together enclose the internal cavity. The front shell 132 can be formed from a light-transmissive material, while the rear shell 136 can be formed from a light-shielding material. This configuration may allow light generated by internal light sources to be directed toward the treatment area while reducing rearward light leakage. The light-shielding rear shell 136 may thereby improve phototherapy efficiency by directing emitted light toward the skin.

The front shell 132 and rear shell 136 can be joined using detachable connection mechanisms. In an embodiment, the detachable connection comprises snap-fit hooks formed on one shell and corresponding receiving apertures formed on the other shell to permit tool-free assembly and disassembly. In an alternative embodiment, magnetic coupling elements such as magnets and ferromagnetic plates can be disposed on the respective front shell 132 and the rear shell 136 . In further embodiment, positioning guide posts having threaded apertures can be provided to receive screws or other threaded fasteners. The detachable connection arrangement facilitates access to internal components for maintenance, battery replacement, or servicing.

In an embodiment, the housing can be configured in various form factors, including but not limited to a handheld device, a jar-cap assembly, a mat structure, or a massager. In the jar-cap configuration, the rolling ball incorporating conductive regions and light-transmissive regions is integrated into a cap structure configured for attachment to a container. In the mat configuration, one or more rolling balls can be mounted within a generally planar housing to facilitate treatment of larger body areas. In the massager configuration, the rolling ball can be incorporated into the device intended for localized or targeted therapeutic application. In handheld device, the housing can include an ergonomic handle portion or an ergonomically contoured body configured to facilitate gripping and manipulation during treatment.

In an embodiment, where the housing is configured as the jar-cap, the housing can include a threaded engagement portion, snap-fit mechanism, or equivalent attachment structure configured to couple with a container, such as a skincare product jar. The rolling ball may be mounted within the cap structure such that, when the cap is inverted and applied to the skin, a portion of the rolling ball protrudes through an opening in the cap for skin contact. The circuit board and battery can be disposed within the cap structure, and a sealing element may be provided between the cap and the container to prevent ingress of topical product into the electronic components. The jar-cap configuration can permit application of a topical formulation followed by rolling of the rolling ball to massage the product into the skin while simultaneously delivering electrical stimulation and phototherapy.

In an embodiment, where the housing is configured as a mat, the housing can comprise a generally planar and optionally flexible substrate having a plurality of rollingballs mounted therein. The rolling balls can be arranged in a grid pattern or other array configuration to provide coverage of larger anatomical regions, such as the back, shoulders, or legs. Each rolling ball can be rotatably mounted within a socket formed in the substrate, with electrical connections routed through the substrate to a centralized circuit board or distributed control electronics. The mat can include a power cord for connection to an external power source or can incorporate a rechargeable battery pack. The mat configuration can permit hands-free treatment, wherein a user rests against the mat, and the rolling balls provide rolling massage, electrical stimulation, and phototherapy to contact areas.

In an embodiment, where the housing is configured as a massager, the rolling ball can be integrated into a handheld or wearable therapeutic device. The massager configuration can further include vibration motors or other mechanical massage mechanisms in addition to the rolling ball, providing electrical stimulation and phototherapy. The device can include an ergonomic handle, strap, or mounting structure configured to position the device against targeted treatment areas. The combination of mechanical massage, rolling contact, electrical stimulation, and phototherapy can provide synergistic therapeutic effects compared to individual modalities used independently.

In an embodiment, the therapeutic device includes a handle extending from the housing to facilitate gripping and manipulation during use. In some implementations, the handle comprises a C-shaped configuration sized to be positioned between two fingers of a user.

The C-shaped handle can define a curved profile forming an opening sized to receive adjacent fingers of a user. The handle can include contoured surfaces configured to conform to finger anatomy, thereby improving ergonomic comfort during extended use.

In an embodiment, the C-shaped handle is configured to be gripped between an index finger and a middle finger. The spacing between opposing curved portions of the handle can correspond to typical anatomical spacing between adjacent fingers, permitting insertion of two fingers into the defined opening. The user can secure the device by curling the fingers around the curved portions during movement across the skin.

The C-shaped handle can provide enhanced control during therapeutic application. Positioning the handle between two fingers can enable fine motor control, allowing the user to direct rolling movement of the rolling ball across targeted areas and to regulate contact pressure between the rolling ball 110 and the skin.

The C-shaped handle can extend from a rear portion of the housing opposite the rolling ball 110. The handle can be integrally formed with the housing or attached via mechanical fasteners or equivalent attachment mechanisms.

The handle can be formed from materials selected to provide structural support and user comfort. In certain embodiments, the handle includes a rigid core and a soft overmold layer disposed on gripping surfaces. In alternative embodiments, the handle can be of unitary construction formed from a material providing sufficient rigidity while maintaining user comfort.

In an embodiment, the rolling ball 110 is rotatably mounted within the housing 100 and formed from a transparent or translucent material selected from glass, crystal, quartz, ceramic, plastic, or equivalent materials. The outer surface of the rolling ball 110 includes conductive regions and at least one light-transmissive region 116. The conductive regions are configured to deliver electrical stimulation, while the light-transmissive region 116 permits transmission of light from an internal phototherapy source.

The transparent material of the rolling ball 110 can comprise glass, quartz, ceramic, or optically clear polymeric materials. Glass and quartz can provide high optical clarity and durability. Transparent or translucent ceramics can permit light transmission while supporting conductive surface treatments. Polymeric materials can permit light transmission and facilitate deposition or embedding of conductive elements.

In an embodiment, the rolling ball material can comprise a translucent stone or mineral material capable of permitting partial light transmission while providing a natural surface texture for skin contact.

The rolling ball material can support patterned application of conductive coatings or embedded conductive elements while preserving intervening light-transmissive regions. This arrangement enables concurrent delivery of electrical stimulation through conductive regions and phototherapy through light-transmissive regions during rolling contact.

The rolling ball 110 can be rotatably mounted within the housing cavity such that rotation occurs during movement across the skin, permitting sequential engagement of conductive and light-transmissive regions with the treatment surface.

In an embodiment, the rolling ball 110 includes at least one electrical stimulation element disposed on its surface and configured to deliver electrical stimulation during rolling contact. Light emitted from an internal or external phototherapy source can pass through the rolling ball 110 simultaneously. Patterned conductive traces provide electrical contact while intervening light-transmissive regions permit phototherapy delivery.

In certain embodiments, the conductive region includes at least one electrical stimulation element configured to deliver stimulation therapy during rolling movement. The electrode can comprise a patterned conductive trace formed on the outer surface of the rolling ball 110 using deposition, plating, sputtering, printing, or equivalent techniques.

The rolling ball 110 can include at least one conductive region implemented as patterned conductive traces or electrode surfaces formed on its outer surface. In an embodiment, the conductive region includes patterned conductive traces 114 arranged as a first patterned conductive trace 114a and a second patterned conductive trace 114b. The patterned conductive traces can be formed from electroplated, sputtered, or otherwise deposited metals such as copper, nickel, silver, gold, or equivalent conductive materials. The patterned conductive traces can define grid patterns, honeycomb geometries, or micro-textured electrode islands.

In an embodiment, the electrical stimulation element can comprise one or more translucent or transparent ceramic spheres, each functioning as an electrode surface. The electrode surfaces can be coated with conductive material, such as gold plating, to form conductive regions. Multi-color LEDs 108 can be positioned behind each rolling ball 110 such that optical radiation passes through the transparent material and exits through the light-transmissive region 116.

In an embodiment, the electrical stimulation element can comprise multiple smaller ceramic rolling elements, each supporting a plated conductive electrode surface. The ceramic rolling elements may reduce sliding friction and enhance rolling contact. The conductive shaft 122 provides an electrical pathway between the conductive regions and the circuit board 120.

In an embodiment, electrical contact interfaces, such as rolling conductive brushes or slip contacts, can be configured to maintain low contact impedance, for example, below approximately 0.1 ohms, without limitation. The rolling ball 110 can be structurally reinforced and configured to withstand impact, drop, and compressive loads without the formation of sharp edges or structural failure.

In an embodiment, the phototherapy unit 118 includes a plurality of LEDs 108, for example, 5050 packaged dual-chip LEDs, configured to emit light at wavelengths of approximately 633 nm ± 2 nm (red light) and 830 nm ± 2.5 nm (near-infrared light). The optical power density may be approximately 30 mW/cm² ± 5 mW/cm², without limitation. The number of optical emission positions may be, for example, 10 or 12, depending on the design configuration.

In an embodiment, the phototherapy unit 118 may emit light at selected therapeutic wavelengths. Red light in a range of approximately 630–660 nm can be utilized to stimulate collagen production, enhance blood circulation, and promote cellular regeneration. Near-infrared light in a range of approximately 830–850 nm may penetrate deeper tissue layers to support pain relief, inflammation reduction, and accelerated healing. Blue light in a range of approximately 415–470 nm may be used for antibacterial treatment and acne reduction. In some embodiments, the phototherapy unit 118 includes LEDs emitting at multiple wavelengths, thereby permitting selection of therapeutic modes according to treatment requirements. The optical power density at the skin surface can range from approximately 10–100 mW/cm², and treatment sessions can last approximately 5–20 minutes, depending on the intended application.

In an embodiment, the conductive regions provide at least one electrical stimulation element disposed on the rolling ball 110. The electrical stimulation element can comprise conductive traces, patterned conductive traces 114, electrodes, or conductive coatings formed on the surface of the rolling ball 110. The conductive material can be applied by electroplating, conductive ink deposition, embedded conductive wire placement, patterned conductive trace attachment, or other suitable metallization or deposition techniques. The conductive regions can be patterned to define discrete electrode areas while maintaining intervening light-transmissive regions substantially free of opaque conductive material, thereby permitting simultaneous electrical stimulation and light transmission through the rolling ball 110.

In an embodiment, the therapeutic device can deliver one or more forms of electrical stimulation therapy, including electrical muscle stimulation (EMS), microcurrent therapy, galvanic therapy, transcutaneous electrical nerve stimulation (TENS), or radiofrequency (RF) therapy. The electrical stimulation elements are electrically coupled to the circuit board 120 within the housing 100 through the conductive shaft 122 or another electrical coupling mechanism, such as conductive pins or compliant contacts, configured to maintain electrical continuity while accommodating rotation of the rolling ball 110.

In an embodiment, the electrical stimulation parameters can be configured according to the selected therapy mode. For microcurrent therapy, current intensity can range from approximately 10–600 µA, with voltage levels of approximately 0–50 V and frequencies of approximately 0.5–10 Hz. For EMS therapy, current intensity can range from approximately 1–80 mA, with pulse widths of approximately 50–400 µs and frequencies of approximately 20–100 Hz. For TENS therapy, current intensity may range from approximately 1–100 mA, with pulse widths of approximately 50–250 µs and frequencies of approximately 2–150 Hz. For galvanic therapy, direct current in a range of approximately 0.1–4 mA may be applied. The circuit board 120 may store multiple preset treatment protocols defining electrical parameters suitable for different therapeutic applications.

In an embodiment, the electrical stimulation element on the rolling ball 110 may be implemented as a patterned conductive trace printed or deposited on the surface of the rolling ball 110, including on patterned conductive traces 114. The patterned conductive trace 114 may comprise sputtered metal, electroplated copper, nickel, gold, or printed conductive ink. The conductive traces are electrically routed through the conductive shaft 122 to the circuit board 120, which is configured to multiplex and modulate the patterned conductive traces for delivery of microcurrent or EMS stimulation while simultaneously controlling operation of the phototherapy unit 118. The patterned conductive trace 114 may be electrically connected to the circuit board 120 through a compliant ring contact or equivalent conductive interface.

In an embodiment, one or more patterned conductive traces 114 are arranged circumferentially around the rolling ball 110. Each patterned conductive trace 114 may extend from a first end region toward a second end region of the rolling ball 110 and connect to the conductive shaft 122. The patterned conductive traces 114 may have a tapered width that is wider at a central circumferential band and narrower toward the ends, or may have a substantially uniform width, to regulate current density distribution. The circuit board 120 may individually address each patterned conductive trace via a switching matrix to energize selected patterned conductive traces in contact with the skin. The circuit board 120 may monitor contact via sensors and adjust current delivery to each patterned conductive trace 114 according to preprogrammed waveforms and/or user input.

In some embodiments, the patterned conductive trace comprises a metal coating applied to the surface of the rolling ball. The metal coating may be formed by electroplating, sputtering, vapor deposition, or other metallization processes, and may comprise silver, gold, copper, or other conductive metals suitable for skin-contact applications. Electroplating may deposit a thin conductive layer onto selected regions of the rolling ball surface while leaving other regions uncoated to preserve light transmission.

In an embodiment, the conductive elements on the surface of the rolling ball 110 may additionally or alternatively be formed using conductive ink deposition, silver plating, or gold plating. Conductive ink may be printed or deposited in predetermined patterns to create conductive traces. Silver or gold plating may be applied through chemical or electrochemical deposition processes to define conductive regions.

In an embodiment, the electrical stimulation element comprises a patterned conductive trace formed by electroplating a metal coating onto the surface of the transparent rolling ball. The electroplating process may deposit a conductive layer onto selected regions while leaving other regions uncoated to define light-transmission areas. The metal coating may comprise silver, gold, copper, or other biocompatible conductive metals. The resulting patterned conductive trace defines discrete electrode areas separated by intervening light-transmissive regions.

In an embodiment, two primary patterned conductive traces, a first patterned conductive trace 114a and a second patterned conductive trace 114b, are formed on the rolling ball 110 and spaced apart circumferentially to define a light-transmissive region 116 therebetween. The phototherapy unit 118 may be positioned within the housing 100 or embedded within the rolling ball 110 to direct therapeutic light through the light-transmissive region 116 toward the skin located between the first and second patterned conductive traces. The circuit board 120 may simultaneously drive the first and second patterned conductive traces as bipolar electrodes. Sensors may confirm proper skin positioning in the inter-strip region and trigger a coordinated light and bipolar stimulation program executed by the circuit board 120.

In an embodiment, the first patterned conductive trace 114a and the second patterned conductive trace 114b may be formed as continuous or segmented circumferential traces on the spherical rolling ball 110. Each strip is electrically routed to the conductive shaft 122 and to the circuit board 120. The two-strip configuration creates an inter-strip optical window forming the light-transmissive region 116, thereby enabling simultaneous bipolar stimulation and phototherapy directed to substantially the same treatment region. The circuit board 120 may selectively energize the strips only when sensors confirm skin contact across the inter-strip region. Because the strips are individually addressable, polarity may be periodically reversed to distribute electrochemical loading across the electrode-skin interface.

In an embodiment, the rolling ball 110 includes a plurality of patterned conductive traces disposed on its outer surface. The patterned conductive traces may comprise grids, rings, linear traces, or raised electrode islands. The patterned conductors define optical windows between adjacent conductive regions to permit light transmission through the rolling ball. The optical windows comprise regions substantially free of opaque conductive material.

In an embodiment, the patterned conductive traces comprise raised electrode islands disposed on the outer surface of the rolling ball 110. The raised electrode islands may protrude slightly from the rolling ball surface to enhance electrical contact with skin during rolling movement. The islands may be spaced apart to define optical windows therebetween for light transmission.

In an embodiment, polarity reversal may occur at predetermined intervals during therapeutic application. The reversal interval may range from approximately 1–60 seconds, for example, 5–15 seconds for microcurrent therapy and 10–30 seconds for galvanic therapy. The circuit board may generate biphasic waveforms having symmetric or asymmetric positive and negative phases, with phase durations of approximately 50–500 µs. In charge-balanced configurations, the total charge delivered during the positive phase may substantially equal that delivered during the negative phase, thereby reducing electrochemical effects at the electrode-skin interface. The polarity reversal interval may be user-adjustable.

In an embodiment, the raised electrode islands may be formed by selective electroplating, in which a thicker conductive layer is deposited in predetermined regions. A mask may define electrode island locations, and electroplating may build the conductive material to a height of approximately 0.1–1.0 mm above the surrounding rolling ball surface. Alternatively, pre-formed conductive elements may be attached using conductive adhesive or mechanical fastening. The electrode islands may have a diameter of approximately 2–10 mm and may be spaced apart by approximately 3–15 mm to define optical windows. The raised profile may enhance electrical contact during rolling over uneven skin surfaces.

In an embodiment, the electrical stimulation elements may be formed by embedding metal wire, such as silver or gold wire, within slots or grooves 104 formed on the surface of the rolling ball 110. The wire may be positioned within the grooves and secured to form microcurrent electrodes that are flush with or slightly raised relative to the rolling ball surface.

In an embodiment, each electrical stimulation element may be configured as a longitudinal patterned conductive trace 114 extending along the surface of the rolling ball. The strip may extend toward opposite ends of the rolling ball 110 and may have a width that decreases from a central region toward the ends. This tapered configuration may provide a larger contact area at the central region of the rolling ball, where skin contact is most frequent during rolling.

In some embodiments, multiple electrical stimulation elements may be arranged along an axial direction of the rolling ball at spaced intervals. The electrodes may be circumferentially distributed around the rolling ball 110 with spacing between adjacent electrodes. An electrode positioned near a central axial region of the rolling ball may have a greater width, while electrodes positioned toward opposite sides may have progressively smaller widths. This variable-width configuration may correspond to the spherical geometry of the rolling ball, providing consistent electrode contact with skin during rotation. Current delivery to the patterned conductive traces 114 may be controlled by the circuit board 120 through the conductive shaft 122.

In an embodiment, the therapeutic device may include one or more massage spheres 130 arranged at intervals relative to one or more therapeutic rolling balls 110. Multiple rolling balls 110 may be rotatably mounted within the housing 100 through respective conductive shafts 122, allowing individual or coordinated control via the circuit board 120. The rolling balls 110 and massage spheres 130 may be proportionally arranged to provide substantially uniform stimulation, irradiation, and massage therapy across the treatment area. Light-transmissive portions of the housing 100 may be aligned with spaces between adjacent massage spheres 130 and rolling balls 110. Multiple light sources may be aligned with these light-transmissive portions to enhance light output. Such alignment may reduce obstruction of emitted light and increase the proportion of light reaching the skin.

In an embodiment, massage spheres 130 may be positioned between rolling balls 110 and configured as universal rolling elements capable of multi-axis rotation. Each massage sphere 130 may rotate freely in response to movement of the therapeutic device in any direction across the skin. This universal rolling configuration may reduce friction and resistance between the device and the skin surface during therapeutic application.

In an embodiment, the massage spheres 130 are positioned between adjacent rolling balls 110 within the housing 100. The spacing between the rolling balls 110 defines mounting locations for the massage spheres 130. During therapeutic application, the massage spheres 130 provide mechanical massage to the skin through rolling contact, delivering mechanical stimulation concurrently with the electrical stimulation therapy and phototherapy provided by the rolling balls 110.

In an embodiment, the universal rolling ball 110 configuration of the massage spheres 130 permits multidirectional movement of the therapeutic device across the skin without requiring reorientation. The massage spheres may accommodate forward, backward, lateral, and diagonal movement. This omnidirectional rolling capability reduces drag forces when the device changes direction, thereby enabling smooth transitions during treatment.

In an embodiment, the device may include one or more stimulation elements selected from a phototherapy element, a microcurrent element, a Peltier element, a vibrational element, a thermal element, an ultrasonic wave therapy element, a magnetotherapy element, an electrical muscle stimulation (EMS) element, a galvanic element, a TENS element, a radiofrequency (RF) element, or a pulsed electromagnetic field (PEMF) element, without limitation. Each stimulation element may operate independently or in combination with other elements to provide multimodal therapy.

In an embodiment, the rolling balls 110 are spaced apart along an arcuate trajectory at predetermined intervals. The spaces between adjacent rolling balls 110 define light-transmission regions through which light from internal light sources may pass for phototherapy delivery. The spacing may be configured to permit sufficient optical transmission while maintaining adequate surface contact area for electrical stimulation therapy.

In an embodiment, the therapeutic device includes a respective phototherapy unit 118 associated with each of at least two rolling balls 110. The number of phototherapy units 118 may correspond to the number of rolling balls 110. Each phototherapy unit 118 may be mounted on a lamp board positioned within the cavity and oriented toward the surface of a corresponding rolling ball 110. Each lamp board may include one or more light sources configured to emit light through light-transmissive regions of the associated rolling ball 110, thereby permitting independent phototherapy delivery through each rolling ball.

In an embodiment, multiple rolling balls 110 are rotatably mounted on a common conductive shaft 122 disposed within the cavity. The common conductive shaft 122 mechanically supports the rolling balls 110 and provides an electrical connection between the circuit board 120 and the electrical stimulation elements disposed on each rolling ball 110. In an alternative embodiment, each rolling ball 110 is rotatably mounted on a separate conductive shaft, each shaft being electrically connected to the circuit board 120.

In an embodiment, the rolling balls 110 are configured to rotate independently during therapeutic application. As the therapeutic device moves across the skin, each rolling ball 110 rotates at a rate corresponding to its movement relative to the skin surface. Independent rotation permits each rolling ball 110 to maintain rolling contact even when the device traverses uneven skin contours or changes direction.

In an embodiment, the stimulation element comprises a microcurrent component configured to provide microcurrent therapy. The microcurrent component may include one or more electrodes disposed on a surface of the housing, such as an end face or connecting surface, and electrically coupled to the internal circuit board. The circuit board is configured to generate controlled low-level electrical pulses delivered through the electrodes to the user’s skin. Microcurrent stimulation may promote circulation, support cellular energy (ATP) production, and enhance skin tone and elasticity. The microcurrent component may operate independently or concurrently with other stimulation elements, such as phototherapy, thermal, or ultrasonic components.

In an embodiment, the stimulation element comprises a heating and cooling component configured to provide controlled thermal therapy. The thermal component may include a Peltier module, a resistive heating element, or another thermoelectric device disposed beneath or adjacent to a light-transmitting plate or treatment surface. The thermal component is thermally coupled to the housing surface to transfer heat or cooling to the user’s skin. The control circuit regulates the direction and magnitude of current supplied to the thermoelectric component to selectively produce heating or cooling effects.

In an embodiment, the stimulation element comprises a magnetotherapy component including one or more electromagnetic coils or permanent magnets configured to generate a pulsed or static magnetic field. The stimulation element may alternatively or additionally comprise an ultrasonic wave therapy component including one or more piezoelectric transducers configured to emit ultrasonic vibrations, for example, in a frequency range of approximately 0.8–3 MHz, to stimulate tissue, enhance transdermal absorption, or relieve muscular tension.

In an embodiment, the phototherapy component provides optical stimulation at selected wavelengths, while the microcurrent component delivers controlled low-level electrical currents through surface electrodes. The heating and cooling components regulate surface temperature to provide thermal therapy. These components may be controlled individually or simultaneously via the circuit board and a user interface, allowing selection of desired therapy modes according to treatment requirements.

In an embodiment, one or more light sources are positioned within the housing 100 and oriented toward the surface of the rolling ball 110. The light sources, such as the phototherapy unit 118, may comprise LEDs 108 configured to emit light at therapeutic wavelengths. Light emitted from the light sources passes through light-transmissive regions of the rolling ball 110 to irradiate the skin during rolling contact. The combination of electrical stimulation and phototherapy delivered through the rolling rolling ball 110 may provide substantially uniform treatment across the skin surface.

In an embodiment, the phototherapy unit 118 is positioned within the cavity of the housing and oriented toward the rolling ball 110. The phototherapy unit may emit light through the light-transmissive region 116 of the rolling ball 110 toward the skin during therapeutic application. The phototherapy unit 118 may be mounted on a lamp board or other support structure and aligned to direct light toward optical windows defined between or adjacent to conductive regions on the rolling ball surface, thereby enabling concurrent phototherapy and electrical stimulation.

In an embodiment, the phototherapy unit comprises at least one LED 108 configured to emit light at one or more therapeutic wavelengths. The LEDs may be mounted on a lamp board positioned within the cavity opposite the rolling ball 110. The lamp board may have a semicircular or arcuate configuration corresponding to the curvature of the rolling ball to maintain a substantially uniform spacing between the LEDs and the rolling ball surface. Multiple LEDs may be distributed along the lamp board to provide even light emission.

In an alternative embodiment, the transparent rolling ball 110 includes a plurality of LEDs 108 integrated within an interior volume of the rolling ball 110. The integrated LEDs may emit light outward through the transparent material of the rolling ball 110 toward the skin. Patterned conductive traces disposed on the outer surface of the rolling ball 110 may provide electrical stimulation while defining optical windows between conductive regions. These optical windows are substantially free of opaque conductive material, permitting light generated by the internal LEDs to exit the rolling ball 110 for phototherapy delivery.

In an embodiment, the circuit board 120 is disposed within the cavity of the housing 100 and electrically connected to at least one electrical stimulation element on the rolling ball 110 and to the phototherapy unit 118. The circuit board 120 coordinates the operation of the electrical stimulation elements and the phototherapy unit to permit simultaneous or sequential delivery of electrical stimulation therapy and phototherapy.

In some embodiments, the circuit board 120 is electrically connected to a power source or battery 126 disposed within the housing cavity. The battery 126 supplies electrical energy to the circuit board 120, which distributes power to the electrical stimulation elements and the phototherapy unit 118. The circuit board 120 may include control circuitry configured to regulate electrical stimulation parameters, including current intensity, voltage, frequency, waveform characteristics, and therapy modes. The circuit board 120 may further regulate phototherapy parameters, including light intensity and activation timing.

In an embodiment, the electrical stimulation element delivers pulsed electrical energy under the control of the circuit board 120. The stimulation waveform may comprise a pulsed biphasic waveform having a pulse width of approximately 50 µs and a frequency of approximately 50 Hz, operating at up to 100% duty cycle, without limitation.

In an embodiment, the maximum output voltage may be approximately 154 mV at 500 Ω, 465 mV at 52 kΩ, and 2.2 V at 10 kΩ. The maximum output current may be approximately 309 µA at 500 Ω, 232 mA at 2 kΩ, and 202 mA at 10 kΩ. The maximum phase charge may be approximately 6.16 µC at 500 Ω, and the maximum current density may be approximately 0.34 mA/cm² at 500 Ω. The maximum power density may be approximately 3.44 µW/cm² at 500 Ω. Output tolerance may be approximately ±10%. All values are exemplary and non-limiting.

In an embodiment, the circuit board 120 provides multiple EMS intensity levels, for example, low, medium, and high, with a default startup mode set to low intensity. Actuation of a user input button 128 may cycle through the available intensity levels. An audible indicator, such as a buzzer, may confirm each mode change.

In an embodiment, the device includes contact-sensing logic, wherein the circuit board 120 monitors electrode current and enables stimulation only when a minimum current threshold of approximately 100 nA is detected. Upon detection of valid skin contact, a white LED indicator is activated to provide user feedback.

In an embodiment, the device is configured for use with a conductive gel that reduces electrical impedance between the rolling ball 110 and the user’s skin. The circuit board 120 dynamically adjusts stimulation amplitude based on real-time impedance measurements to maintain a target therapeutic output.

In an embodiment, the device includes an automatic safety timer, wherein the circuit board 120 automatically powers the device OFF after approximately four (4) minutes of continuous operation, without limitation. The device further includes a low-battery indicator, wherein one or more LEDs are illuminated when battery voltage falls below a predefined threshold.

In an embodiment, the circuit board 120 performs periodic polarity reversal to reduce or prevent electrochemical skin irritation. A reversal schedule, ramp duration, and dwell time may be stored in firmware and dynamically adjusted based on sensor feedback. The circuit board 120 may further record usage logs, including therapy duration, energy delivered, selected mode, and detected fault conditions, in onboard non-volatile memory. The logs may be accessed via wired or wireless communication interfaces.

In a preferred embodiment, a first patterned conductive trace 114a and a second patterned conductive trace 114b are circumferentially spaced apart on the rolling ball 110 to define an intervening light-transmissive region 116. The circuit board 120 drives the first patterned conductive trace 114a and the second patterned conductive trace 114b as bipolar electrodes for electrical stimulation delivery using one or more selectable waveform generators. A phototherapy unit 118 comprising one or more LED modules 108 disposed within the housing 100 and/or within the rolling ball 110 is configured to irradiate a skin region positioned between the first patterned conductive trace 114a and the second patterned conductive trace 114b through the light-transmissive region 116 while bipolar stimulation is applied across the strips.

In an embodiment, the therapeutic device includes one or more user input mechanisms, such as a button 128, electrically coupled to the circuit board 120 and configured to implement multi-press, press-and-hold, and press-sequence recognition to enable compact physical control without requiring a paired mobile device.

The circuit board 120 executes firmware configured to debounce button inputs, detect press duration, and identify press sequences within a configurable inter-press window (for example, approximately 400–700 ms). A short press (e.g., less than 0.5 seconds) cycles through therapy intensity levels (e.g., low, medium, high) when the device is ON, with each short press incrementing the level and generating user feedback such as an audible tone or LED indication. If the device is in standby, a short press may wake the device without initiating stimulation until contact sensors confirm valid skin contact.

A press-and-hold (e.g., approximately 1.5–2.0 seconds or longer), defined in firmware, powers the device ON and initiates a default therapy profile (e.g., combined electrical stimulation and phototherapy), thereby reducing accidental activation. A double press within a detection window (e.g., approximately 600 ms) toggles electrical stimulation OFF while maintaining the state of the phototherapy unit 118. If stimulation is already OFF, the double press re-enables stimulation. A triple press within the detection window toggles the phototherapy unit 118 OFF while maintaining electrical stimulation.

The circuit board 120 stores locally selected modes in non-volatile memory such that subsequent sessions retain the selected configuration unless changed. A long press (e.g., ≥ 3.0 seconds) powers the device OFF, terminates active outputs in a controlled manner (including ramp-down of stimulation and LED output), logs session completion, and transitions the device into a low-power state.

Before enabling stimulation or phototherapy outputs, the circuit board 120 validates contact sensing using one or more capacitive, impedance, or pressure sensors. If contact thresholds are not satisfied, output activation or toggling commands are ignored, and status feedback is provided. Firmware further implements software debouncing (e.g., approximately 20–50 ms), anti-ghosting logic, and configurable press-window parameters. Mode changes triggered by button 128 may be time-stamped and appended to usage logs stored in onboard non-volatile memory and accessible via wired or wireless communication, including optional synchronization to a cloud profile.

In an embodiment, the therapeutic device comprises a low-power wireless transceiver integrated with the circuit board 120, such as a Bluetooth® Low Energy (BLE) module, configured to support secure pairing with a smartphone application and optional cloud services. The circuit board 120 executes a communication stack enabling device advertisement, pairing, and encrypted data exchange.

User profiles stored within the application may include metadata such as age bracket, skin type, preferred modes, and therapy presets (e.g., stimulation intensity, phototherapy wavelength selection, and session duration). Upon initial pairing, the circuit board 120 securely exchanges a device token with the application and stores selected profile parameters in non-volatile memory. Multiple user profiles may be stored locally and selected via the application or predefined button sequences.

The circuit board 120 logs session events, including start time, stop time, mode changes, impedance values, energy delivered, battery voltage, and fault codes, together with timestamps. When paired, a real-time clock (RTC) reference may be obtained from the smartphone, and logs may be uploaded to a cloud service via authenticated and encrypted communication. Cloud-stored logs may include server timestamps, device identifiers, and optionally anonymized telemetry data. Users may opt in or opt out of cloud synchronization via the application.

Adaptive presets may be generated by the application based on telemetry data and pushed to the circuit board 120, subject to device-enforced safety limits, including maximum current and maximum runtime constraints. All communications between the device and the application are encrypted.

In an embodiment, a live mode is provided in which the application displays real-time impedance, stimulation intensity, and LED status during an active session via authenticated BLE communication. In clinician-enabled configurations, consent-based telemetry sharing may be supported through the cloud platform.

The circuit board 120 may support secure over-the-air (OTA) firmware updates delivered through the application. Update packages are authenticated and verified before installation. During an OTA update, therapeutic outputs are suspended until successful completion. The cloud platform may store user preferences and scheduling reminders, while the device operates autonomously using the most recently synchronized presets and locally enforced safety logic when disconnected.

Remote commands requiring elevated privileges (e.g., disabling therapy) are subject to local hardware-based safety checks and explicit user confirmation within the application.

In an embodiment, the housing 100 comprises a USB Type-C charging interface and optionally a wireless charging receiver coil. Charging is managed by a battery management subsystem under control of the circuit board 120 and an onboard charge controller integrated circuit (IC). The Type-C interface is electrically coupled to the charging IC and the battery 126.

The circuit board 120 monitors charging state, battery temperature, cell voltage, and charging current via analog-to-digital converter (ADC) channels and thermistor inputs. The charging IC implements constant-current/constant-voltage charging for the lithium-ion battery 126 and supports charge termination and maintenance charging as required.

When a wired charging connection is detected, the circuit board 120 disables therapeutic outputs, verifies safety thresholds, and initiates a charging routine. In the event of a fault condition, including over-temperature or over-current, charging is suspended and the fault is logged. A visual indicator communicates charging status.

In embodiments including wireless charging, a wireless power receiver IC supplies regulated current to the charging IC. Upon detection of a valid wireless charging field, the circuit board 120 applies the same monitoring and safety procedures as with wired charging. Optional foreign-object detection may be implemented to terminate charging upon detection of unsafe conditions. The circuit board 120 may record charge cycle counts and estimate battery health, with such metrics stored locally and optionally synchronized to the cloud.

In certain embodiments, the circuit board 120 may further adjust stimulation or phototherapy parameters based on detected application of topical agents, such as serums or creams, as inferred from impedance characteristics or sensor feedback.

In an embodiment, one or more patterned conductive traces 114 and associated conductive traces disposed on the rolling ball 110 receive electrical power from internal circuitry within the housing 100 via the conductive shaft 122. Electrical connections may be routed through the structure of the rolling ball 110 to supply power to surface-mounted conductive elements.

In an embodiment, an electrical connection between the circuit board 120 and the conductive elements on the rolling ball 110 is established through the conductive shaft 122. The conductive shaft 122 is positioned within the housing 100 and supports rotational mounting of the rolling ball 110. The conductive shaft 122 provides an electrical pathway from the circuit board 120 to one or more electrical stimulation elements disposed on the rolling ball 110 while permitting rotation during therapeutic use.

In an embodiment, conductive needles are positioned at respective ends of the conductive shaft 122 to establish electrical contact with the circuit board 120. The conductive needles may be electrically connected to the circuit board 120 via wires or conductive traces. This arrangement reduces or eliminates direct-wired connections to the rotating shaft, thereby reducing wear and tangling during rotation.

In an embodiment, the circuit board 120 includes a control interface electrically connected to button 128 positioned on the housing 100 for activating and deactivating the device. The circuit board 120 further includes a charging interface electrically connected to the battery 126 disposed within the housing cavity.

In an embodiment, the conductive shaft 122 is disposed within the housing cavity and rotatably supports the rolling ball 110. The rolling ball 110 is mounted on the conductive shaft 122 such that the rolling ball rotates freely relative to the shaft during movement across skin tissue.

In an embodiment, the conductive shaft 122 is clamped between a front shell 132 and a middle shell 134 of the housing. The front shell 132 and middle shell 134 cooperate to secure the conductive shaft 122 in a fixed position while permitting rotation of the rolling ball 110 mounted thereon. The front shell 132 may include first mounting slots, and the middle shell 134 may include corresponding second mounting slots, with respective ends of the conductive shaft received therein to provide secure clamping.

In an embodiment, at least one electrical stimulation element disposed on the rolling ball 110 is electrically connected to the circuit board 120 via the conductive shaft 122. The conductive shaft 122 provides an electrical pathway between the circuit board 120 and conductive traces or electrodes on the ball surface, permitting transmission of stimulation signals during rotation.

In an embodiment, conductive needles positioned on the middle shell 134 establish electrical contact between respective ends of the conductive shaft 122 and the circuit board 120. Each conductive needle has a first end in electrical contact with the conductive shaft 122 and a second end electrically connected to the circuit board 120.

In an embodiment, the conductive needle arrangement reduces or eliminates wire winding that could otherwise occur if wires were directly connected to the rotating conductive shaft 122. The conductive needles maintain sliding or contact-based electrical engagement with the conductive shaft 122 during rotation, thereby maintaining continuous electrical connectivity between the circuit board 120 and one or more patterned conductive traces 114 during therapeutic use.

In an embodiment, one or more electrical stimulation elements, conductive traces, or patterned conductive traces 114 disposed on the rolling ball 110 may be configured to cooperate with one or more therapeutic elements, including a phototherapy element, microcurrent element, Peltier element, vibrational element, thermal element, ultrasonic wave therapy element, magnetotherapy element, galvanic element, transcutaneous electrical nerve stimulation (TENS) element, radiofrequency (RF) element, pulsed electromagnetic field (PEMF) element, or combinations thereof.

Patterned conductive traces (e.g., hexagonal or honeycomb configurations) on the surface of the rolling ball 110 may deliver electrical stimulation to skin tissue as the ball rolls along the skin, enabling therapeutic current flow during rolling contact.

In another embodiment, the electrical stimulation element is configured to provide electrical muscle stimulation (EMS) therapy. EMS therapy involves delivering electrical impulses to the skin tissue to stimulate underlying muscle tissue. The patterned conductive traces may sequentially contact the skin as the rolling ball 110 rolls, delivering stimulation to different portions of the treatment area and inducing muscle contractions that may tone or strengthen targeted muscle groups.

In another embodiment, the electrical stimulation element is configured to deliver microcurrent therapy. Microcurrent therapy involves the application of low-intensity electrical currents to skin tissue, typically at levels below the threshold of sensory perception. The microcurrent delivered through the patterned conductive traces disposed on the outer surface of the rolling ball 110 may influence cellular activity within the skin. During use, rotation of the rolling ball 110 causes different portions of the patterned conductive traces to sequentially contact the skin, thereby distributing the microcurrent therapy across the treatment area.

In another embodiment, the electrical stimulation element is configured to provide galvanic therapy. Galvanic therapy involves the delivery of direct current to the skin through the patterned conductive traces on the surface of the rolling ball 110. The applied direct current may facilitate transdermal movement of charged molecules into or out of the skin. As the rolling ball 110 rolls along the skin surface, galvanic current is delivered through those conductive traces that are in contact with the skin during rotation.

In another embodiment, the electrical stimulation element is configured to provide transcutaneous electrical nerve stimulation (TENS) therapy. TENS therapy involves the delivery of electrical pulses to the skin to stimulate underlying sensory nerve fibers. The pulses delivered through the patterned conductive traces may modulate pain signals or produce other therapeutic effects. As the rolling ball 110 rolls along the skin, the conductive traces sequentially contact the skin surface and deliver TENS pulses during each contact interval.

In another embodiment, the electrical stimulation element is configured to deliver radio frequency (RF) energy. In embodiments utilizing radiofrequency (RF) energy, the electrical stimulation elements may be configured as bipolar RF delivery electrodes, and the device may include an RF generator configured to deliver energy within a frequency range of approximately 300 kHz to 10 MHz, with current confined between adjacent patterned conductive traces to provide localized dermal heating.

The device may further include one or more contact detection sensors configured to detect physical engagement between the rotating conductive rolling ball 110 and a user’s skin. The control circuitry may dynamically activate only those electrical stimulation elements determined to be in contact with the skin, while deactivating non-contacting elements. Such selective activation may occur in real time during rotation of the ball, thereby improving stimulation precision, reducing unintended current dispersion, and conserving power.

In an embodiment, the circuit board 120 is configured to periodically reverse the polarity of electrical stimulation provided by at least one electrical stimulation element. Polarity reversal involves alternating the direction of electrical current flow through the electrode at predetermined time intervals during therapeutic application. The circuit board 120 switches the electrical connections such that an electrode previously operating as an anode becomes a cathode, and vice versa. The polarity reversal may be implemented using switching circuitry integrated on the circuit board 120.

In an embodiment, the control circuit periodically reverses the polarity of electrical stimulation delivered through the patterned conductive traces disposed on the outer surface of the rolling ball 110. The patterned conductive traces function as electrodes for delivering therapy, and the control circuit alternates the polarity applied to pairs or groups of conductive traces during treatment.

In an embodiment, the circuit board 120 supports controlled polarity reversal between a first patterned conductive trace 114a and a second patterned conductive trace 114b. Reversal may be implemented by periodically swapping which strip is driven anodic and which is driven cathodic, for example, at fixed intervals or per treatment sub-cycle. The circuit board 120 may generate pulsed biphasic waveforms, for example, having a pulse width of approximately 50 µs and a frequency of approximately 50 Hz, with a selectable duty cycle to maintain charge balance. Polarity reversal timing, ramp profiles, and dwell periods may be stored in firmware and adaptively adjusted based on sensed parameters such as contact impedance or temperature.

In an embodiment, polarity reversal occurs at predetermined time intervals during therapeutic application. The reversal interval may range from 1 to 60 seconds, with typical intervals of 5–15 seconds for microcurrent therapy and 10–30 seconds for galvanic therapy. In some cases, the circuit board 120 generates biphasic waveforms having symmetric or asymmetric positive and negative phases, with phase durations in the range of 50–500 µs. The waveform may be charge-balanced such that the total charge delivered during the positive phase substantially equals the total charge delivered during the negative phase, thereby minimizing electrochemical effects at the electrode–skin interface. The polarity reversal timing may be user-adjustable via a control interface or companion application.

Periodic polarity reversal may reduce irritation at the electrode–skin interface. Under constant direct current stimulation, one electrode may experience greater pH change or ion accumulation, potentially leading to localized irritation. Alternating the polarity balances anodic and cathodic exposure across the electrodes, thereby mitigating localized electrochemical effects.

Polarity reversal may also provide a more uniform perceived sensation and treatment coverage. During unidirectional current flow, users may perceive stronger stimulation near one electrode. Alternating polarity averages the perceived stimulation intensity between electrodes, thereby improving uniformity across the treatment area.

Additionally, periodic polarity reversal may reduce electrode polarization and impedance drift. Continuous unidirectional current may cause gradual changes in electrode–skin interface impedance, affecting perceived intensity. Alternating polarity stabilizes the interface by reducing the accumulation of electrochemical byproducts, thereby maintaining more consistent stimulation throughout the treatment session.

In an embodiment, the control circuit implements inherently biphasic stimulation waveforms that provide polarity reversal within each stimulation cycle. Each pulse includes both positive and negative phases, delivering charge-balanced stimulation on a cycle-by-cycle basis. Such biphasic waveforms may be applied to one or more electrical stimulation elements or to the patterned conductive traces disposed on the surface of the rolling ball 110.

In an embodiment, two rolling balls 110 are arranged along an arc trajectory within the housing 100. The arc trajectory corresponds to a curved alignment that follows natural skin contours, enabling both rolling balls to simultaneously contact the skin during therapeutic application. The spacing between the rolling balls along the arc may be selected to optimize treatment area coverage during device movement.

The foregoing embodiments are presented as alternative implementations. The device may be configured as a single-ball handheld unit, a dual-ball arcuate configuration, a flexible housing configuration, a jar-mounted applicator, or a mat-based therapeutic surface, without departing from the scope of the invention.

Arrangement of multiple rolling balls along the arc trajectory increases the total contact area between the device and the skin. With at least two rolling balls simultaneously contacting the skin, a larger treatment region may receive electrical stimulation and phototherapy during each pass across the skin surface.

The multi-ball configuration may enhance treatment effectiveness by providing a more uniform distribution of therapy. Each rolling ball delivers electrical stimulation through its respective conductive regions, and the combined effect improves coverage across the treatment area. Phototherapy delivered through the light-transmissive regions of the respective balls similarly increases optical coverage.

In an embodiment, the therapeutic device includes a handle extending from the housing 100 to facilitate ergonomic gripping and manipulation during treatment. The housing 100 may be configured as a C-shaped handle to improve comfort and control during use.

In an embodiment, the rolling ball 110 and optional massage spheres 130 are mounted using a floating structure configured to adapt to variations in body contour. The floating structure may include springs connecting the rolling ball 110 and massage spheres 130 to a middle shell 134 or other internal support structure within the housing. In some implementations, the springs are formed from conductive material, thereby providing both mechanical support and an electrical pathway between the circuit board 120 and the electrical stimulation elements disposed on the rolling ball 110.

The floating structure permits vertical movement of the rolling ball 110 and massage spheres 130 relative to the housing 100 in response to externally applied force during treatment. When contacting the skin, the ball or massage sphere may compress the associated spring, allowing displacement and improved conformity to the skin contour. Elastic expansion and contraction of the springs enable the components to “float” relative to the housing, thereby increasing contact consistency with the skin surface.

The floating mount structure permits adaptation to body areas having varying surface geometries, such as the face, jaw, and neck. Each rolling ball and massage sphere may independently adjust position in response to localized contours. Springs associated with regions contacting more prominent skin areas may compress to a greater degree than those contacting recessed regions, thereby maintaining consistent engagement across uneven surfaces.

In an embodiment, the housing 100 is formed from a flexible material configured to permit bending under externally applied force. The flexible material may comprise a polymeric material, such as polyurethane (PU), thermoplastic polyurethane (TPU), or another elastomeric material having sufficient flexibility to enable deformation during therapeutic application. The flexible housing allows massage spheres 130 and rolling balls 110 positioned on opposing sides of the housing 100 to move relative to one another as the housing bends.

In an embodiment, bending of the flexible housing 100 enables the therapeutic device to adapt to skin regions having varying contours or degrees of depression. When the housing bends, massage spheres 130 and rolling balls 110 located on a first side of the housing may shift relative to massage spheres 130 and rolling balls 110 located on an opposing side. The bending angle of the housing may be adjusted by application of external force, thereby repositioning the massage spheres 130 and rolling balls 110 to conform to concave, convex, or irregular skin surfaces during therapeutic application. The flexible housing 100 may thereby permit partial wrapping of the device around curved body regions.

In an embodiment, inward bending of the flexible housing 100 causes massage spheres 130 and rolling balls 110 on opposing sides to move closer together. This configuration may facilitate contact with concave regions or relatively narrow body contours. Conversely, outward bending of the flexible housing 100 may increase the spacing between massage spheres 130 and rolling balls 110 on opposing sides, thereby accommodating wider body regions or convex contours. The adjustable spacing resulting from housing flexibility improves conformity and contact consistency across different treatment areas.

In an embodiment, the therapeutic device includes a skin detection head disposed on the housing 100. The skin detection head is configured to detect one or more skin quality parameters during therapeutic application. The skin detection head may include one or more sensors capable of measuring characteristics of skin tissue, thereby enabling assessment of skin condition before or during treatment.

In an embodiment, the therapeutic device includes a detection probe disposed on the housing 100 and electrically coupled to the circuit board 120. The detection probe is configured to detect skin characteristics and generate corresponding electrical signals indicative of detected skin conditions. The circuit board 120 receives and processes the signals to determine one or more skin quality parameters.

In an embodiment, the skin detection head or detection probe is configured to measure parameters including, but not limited to, skin impedance, moisture level, temperature, elasticity, or surface texture. The detected parameters provide information regarding the condition of the skin within the treatment area, thereby enabling dynamic adjustment of therapeutic output based on measured skin characteristics.

In an embodiment, the therapeutic device is configured to adjust one or more electrical stimulation parameters based on detected skin quality. The circuit board 120 may regulate a voltage applied to the electrical stimulation elements in response to measured skin parameters. The voltage may be increased or decreased to maintain a target current density, comfort threshold, or therapeutic range appropriate for the detected skin condition.

In an embodiment, the circuit board 120 adjusts a treatment duration in response to detected skin quality parameters. The duration of a therapeutic session may be extended or shortened based on the detected skin condition, thereby providing treatment intervals appropriate to different skin characteristics.

In an embodiment, the circuit board 120 regulates the current intensity delivered through the electrical stimulation elements based on signals received from the skin detection head or detection probe. The current intensity may be increased or decreased in response to detected skin impedance or related parameters, thereby maintaining consistent therapeutic delivery and user comfort.

In an embodiment, the circuit board 120 regulates the output intensity of the LEDs 108 or other phototherapy elements based on detected skin parameters. The light intensity may be adjusted to increase or decrease optical power delivered to the skin, thereby adapting phototherapy output to the measured skin condition.

In an embodiment, adjustment of treatment parameters based on detected skin quality enables adaptive therapeutic operation. The skin detection head or detection probe may continuously or periodically monitor skin parameters during therapeutic application. The circuit board 120 may dynamically adjust voltage, current intensity, waveform characteristics, treatment duration, and/or light intensity in response to changes in detected skin condition. This adaptive control may improve treatment efficacy, enhance user comfort, and reduce the likelihood of overexposure or irritation.

In some embodiments, a method of providing therapeutic treatment comprises delivering stimulation therapy to the skin through at least one electrical stimulation element while the rolling ball 110 rolls along the skin surface. Rotation of the rolling ball 110 causes different portions of the patterned conductive traces disposed on the outer surface of the ball to sequentially contact the skin, thereby distributing electrical stimulation across a treatment area.

The stimulation therapy delivered during rolling movement may comprise one or more of: phototherapy, microcurrent therapy, galvanic therapy, transcutaneous electrical nerve stimulation (TENS), radio frequency (RF) therapy, pulsed electromagnetic field (PEMF) therapy, thermal therapy, Peltier-based temperature modulation, vibrational therapy, ultrasonic wave therapy, magnetotherapy, or combinations thereof.

In some embodiments, the method further comprises simultaneously delivering light therapy through the light-transmissive region 116 of the rolling ball 110 while delivering electrical stimulation therapy through at least one electrical stimulation element. Accordingly, combined electrotherapy and phototherapy may be administered concurrently as the rolling ball 110 rolls along the skin surface.

As previously described with respect to contact-based selective activation, the control circuitry may independently regulate each patterned conductive trace 114 to ensure that electrical stimulation is delivered only through skin-contacting regions of the rolling ball 110.

Selective activation may occur continuously during the rolling movement of the rolling ball 110. The circuit board 120 may repeatedly update the activation state of individual electrical stimulation elements as the ball rotates and different electrodes sequentially come into and out of contact with the skin surface. This dynamic control may reduce unintended stimulation, improve energy efficiency, and enhance user safety.

In an embodiment, the method further comprises periodically reversing the polarity of the electrical stimulation therapy during treatment. The method may include delivering electrical stimulation at a first polarity for a first time-interval, followed by reversing polarity and delivering electrical stimulation at a second polarity for a second time interval. The polarity reversal may be repeated throughout the treatment session at predetermined or adaptively controlled intervals.

In some embodiments, polarity reversal is implemented between at least two patterned conductive traces 114, such that a first patterned conductive trace 114a operates at the first polarity while a second patterned conductive trace 114b operates at the second polarity during the first time interval, and the polarities of the first patterned conductive trace 114a and the second patterned conductive trace 114b are exchanged during the subsequent time interval. The alternating polarity may be implemented using direct current reversal or biphasic waveform generation to provide charge-balanced stimulation.

In an embodiment, the therapeutic device includes a circuit board 120 configured to selectively activate only those electrical stimulation elements and portions of the phototherapy unit 118 that are in contact with the skin during use, as detected by one or more proximity or contact sensors. The circuit board 120 is disposed within the cavity of the housing 100 and is electrically coupled to the electrical stimulation elements and the phototherapy unit 118.

The circuit board 120 receives sensor output indicative of which portions of the rolling ball 110 are in contact with skin tissue and activates only those electrical stimulation elements and phototherapy elements positioned to deliver therapy to the contacted skin surface. Such selective activation conserves energy by preventing power delivery to electrodes and light sources that are not engaged with the skin during therapeutic application.

In an embodiment, the phototherapy unit 118 and the electrical stimulation elements are configured to activate only upon detection of skin contact. The circuit board 120 detects contact between the surface of the rolling ball 110 and the skin and, in response, energizes the corresponding electrical stimulation elements and light-emitting diodes (LEDs) 108.

When a portion of the rolling ball 110 rotates out of contact with the skin, the circuit board 120 deactivates the electrical stimulation elements and LEDs associated with that portion. This contact-based activation reduces power consumption by limiting energy delivery to elements actively engaged with the skin.

In an embodiment, the rolling ball 110 includes a plurality of independently addressable conductive paths arranged in a pattern on its outer surface. Each conductive path corresponds to a defined surface region of the rolling ball 110 and is configured for independent activation. The circuit board 120 selectively energizes a given conductive path when the associated surface region is detected to be in contact with the skin.

This segmented activation permits targeted delivery of electrical stimulation to specific contact regions while maintaining non-contacting conductive paths in a deactivated state to conserve energy and reduce unintended stimulation.

In an embodiment, a method of providing therapeutic treatment to the skin comprises rolling the rolling ball 110 along the skin surface. The rolling ball 110 includes at least one electrical stimulation element disposed on its outer surface. During rolling movement, rotation of the rolling ball 110 causes the at least one electrical stimulation element to sequentially contact the skin surface, thereby delivering electrical stimulation therapy to the underlying skin tissue.

As the rolling ball 110 rolls along the skin, light passes through the light-transmissive region 116 and irradiates the skin concurrently with electrical stimulation delivered through the at least one electrical stimulation element. The simultaneous delivery of electrical stimulation therapy and phototherapy may occur continuously during the rolling movement of the rolling ball 110 across the skin surface.

In an embodiment, the method comprises moving a therapeutic device containing the rolling ball 110 across the skin surface in a rolling motion. The rolling motion causes the rolling ball 110 to rotate within the housing 100, such that different surface regions of the ball sequentially contact the skin during rotation. As the rolling ball rotates, patterned conductive traces disposed on the outer surface of the rolling ball 110 contact the skin and deliver electrical stimulation therapy, while the light-transmissive regions permit light to pass through the rolling ball 110 for phototherapy delivery.

In an embodiment, simultaneous delivery of electrical stimulation therapy and phototherapy through the rolling ball 110 provides a combined therapeutic treatment during a single rolling pass of the device across the skin. The electrical stimulation therapy may comprise one or more of microcurrent therapy, galvanic therapy, transcutaneous electrical nerve stimulation (TENS), radio frequency (RF) therapy, or combinations thereof.

The light therapy delivered through the light-transmissive region 116 may comprise LED light emitted at one or more therapeutic wavelengths suitable for dermatological or cosmetic applications. The combined delivery of electrotherapy and phototherapy enables treatment using multiple therapeutic modalities during the rolling movement of the rolling ball 110 along the skin surface.

In an embodiment, during therapeutic use, the device operates through coordinated interaction of multiple components to deliver combined electrotherapy and phototherapy. The housing 100 provides structural support and defines a cavity within which the rolling ball 110, circuit board 120, battery 126, and phototherapy unit 118 are disposed. The housing 100 maintains spatial alignment of the components, positions the rolling ball 110 for skin engagement through the opening 102, and encloses the circuit board 120 and phototherapy unit 118 within a protected internal volume.

In an embodiment, the rolling ball 110 rotates within the housing 100 as the device moves across the skin. Rotation of the rolling ball 110 causes different portions of the rolling ball surface to sequentially engage the skin during therapeutic application. Electrical stimulation elements disposed on the ball surface transition between contact and non-contact states during rotation. Each electrode, for example, a patterned conductive trace 114, contacts the skin for a portion of the rotation cycle, delivers electrical stimulation during the contact interval, and subsequently rotates away from the skin as the rolling ball continues to turn.

In an embodiment, the circuit board 120 coordinates the operation of the electrical stimulation elements and the phototherapy unit 118. The circuit board 120 receives electrical power from the battery 126 and distributes power to the electrical stimulation elements via the conductive shaft 122 or another electrical coupling mechanism. The circuit board 120 also supplies power to the phototherapy unit 118 to enable emission of light toward the rolling ball 110.

The circuit board 120 may regulate electrical stimulation parameters, including voltage, current, waveform, frequency, and polarity, and may control light output parameters, including intensity and timing, according to programmed treatment protocols or user-selected settings.

In an embodiment, the circuit board 120 monitors the operational state of the device and selectively activates components based on detected skin contact. When contact between a surface region of the rolling ball 110 and the skin is detected, the circuit board 120 activates the corresponding electrical stimulation elements and associated portions of the phototherapy unit 118. Components associated with non-contacting regions may remain deactivated, thereby conserving energy and reducing unintended exposure.

The circuit board 120 may continuously update activation states as the rolling ball 110 rotates and different surface regions sequentially come into and out of contact with the skin.

In use, the rolling ball 110 may be rolled across the treatment surface while the device concurrently delivers electrical stimulation and phototherapy through contacting patterned conductive traces 114 and light-transmissive regions 116, respectively.

The arrangement of conductive regions and adjacent light-transmissive regions permits electrotherapy and phototherapy to be delivered substantially simultaneously during engagement of the rolling ball 110 with the skin surface.

In an embodiment, the spherical geometry of the rolling ball 110 promotes uniform treatment across different skin regions. The curved surface of the rolling ball 110 maintains consistent contact with the skin during rolling movement, regardless of the orientation of the device relative to the body. The contact area between the rolling ball 110 and the skin remains substantially consistent across different treatment positions, as the spherical surface conforms to local contours during rotation.

This consistent contact geometry facilitates uniform delivery of electrical stimulation and phototherapy across the treatment area and reduces variations in intensity that could otherwise result from inconsistent electrode–skin engagement.

In an embodiment, rotation of the rolling ball 110 distributes therapeutic treatment across the skin surface during movement of the device. As the device is translated over the skin, the rolling ball 110 rotates such that different electrical stimulation elements sequentially contact the skin. Each electrode delivers electrical stimulation during its respective contact interval, and the sequential engagement of multiple electrodes distributes electrotherapy across the treatment area.

Phototherapy delivered through the light-transmissive regions is similarly distributed as the rolling ball 110 rotates, with light passing through different portions of the ball surface during rotation, thereby providing spatially distributed optical irradiation.

In an embodiment, the interaction between the housing 100 and the rolling ball 110 permits continuous rolling contact during therapeutic use. The housing 100 supports the rolling ball 110 via the conductive shaft 122 or another mounting mechanism while permitting free rotation. The opening 102 in the housing 100 exposes a portion of the rolling ball 110 for engagement with the skin, while enclosing and protecting internal components.

The mounting arrangement enables smooth rotation of the rolling ball 110 as the device moves across the skin, thereby maintaining rolling contact rather than sliding contact between the ball surface and the skin. This rolling interaction reduces frictional resistance and improves user comfort during treatment.

In an embodiment in which a plurality of light-emitting diodes (LEDs) 108 are integrated within the rolling ball 110, the ball may comprise a hollow spherical shell defining an internal cavity. The LEDs 108 may be mounted on a central hub structure disposed within the cavity, with radial support members extending toward the inner surface of the ball to position the LEDs relative to the light-transmissive regions.

Electrical power may be supplied to the LEDs through a slip ring assembly, conductive bearing, or equivalent rotating electrical interface that maintains electrical continuity during rotation. In alternative implementations, wireless power transfer may be employed, wherein a receiving coil disposed within the rolling ball receives energy from a transmitting coil positioned within the housing 100.

The integrated LEDs are low-power devices to minimize heat generation, and the transparent or translucent rolling ball material facilitates passive thermal dissipation by conducting heat toward the outer surface, where it is dissipated during rolling contact with the skin.

In an embodiment, an electrical connection between the patterned conductive traces disposed on the outer surface of the rolling ball 110 and the conductive shaft 122 is established through a slip ring, brush contact mechanism, conductive bushing, or conductive bearing assembly. The patterned conductive traces extend from the outer surface toward an internal contact region adjacent to the mounting axis.

A conductive interface element maintains electrical contact between the conductive traces and the conductive shaft while permitting rotation of the rolling ball 110. In certain implementations, spring-loaded brush contacts engage conductive rings formed on an inner surface of the ball adjacent to the rotational axis. The conductive rings are electrically coupled to the patterned conductive traces through internal conductors routed within or along the structure of the rolling ball.

In an embodiment, the conductive shaft 122 includes an external threaded portion, and a locking member comprises a lock nut having a corresponding internal thread. The locking member is threadably engaged with the conductive shaft to secure the rolling ball 110 in position. This threaded locking arrangement enhances mechanical stability and prevents axial displacement or positional shift of the rolling ball during rotation.

In an embodiment, the phototherapy unit 118 is configured to emit light at selected therapeutic wavelengths. For example, red light in a range of approximately 630–660 nm is used to promote collagen synthesis and improve circulation; near-infrared light in a range of approximately 830–850 nm penetrates deeper tissue layers for pain relief and anti-inflammatory effects; and blue light in a range of approximately 415–470 nm provides antibacterial effects and supports acne treatment.

The phototherapy unit 118 includes LEDs emitting at multiple wavelengths, enabling selectable or combinational treatment modes. Optical power density at the skin surface is in a range of approximately 10–100 mW/cm², without limitation.

In an embodiment, the electrical stimulation parameters are configurable according to a selected therapy mode. For microcurrent therapy, the current intensity is in a range of approximately 10–600 µA, with voltage levels of up to approximately 50 V and frequencies of approximately 0.5–10 Hz. For electrical muscle stimulation (EMS), the current intensity is in a range of approximately 1–80 mA, with pulse widths of approximately 50–400 µs and frequencies of approximately 20–100 Hz. For transcutaneous electrical nerve stimulation (TENS), the current intensity is in a range of approximately 1–100 mA, with pulse widths of approximately 50–250 µs and frequencies of approximately 2–150 Hz. For galvanic therapy, direct current in a range of approximately 0.1–4 mA is applied.

The circuit board 120 stores a plurality of predefined treatment protocols, each comprising predetermined electrical parameters corresponding to respective therapeutic applications, and selectively implements one or more of the protocols in response to user input or programmed control.

In an embodiment, raised electrode islands are formed by selective electroplating, wherein a thicker conductive layer is deposited in predefined regions of the ball surface. A masking process defines electrode locations, and electroplating builds the conductive material to a height of approximately 0.1–1.0 mm above the surrounding surface.

Alternatively, pre-formed conductive elements are affixed to the ball surface using conductive adhesive or mechanical fastening. The raised electrode islands have a diameter or width of approximately 2–10 mm and are spaced apart by approximately 3–15 mm to define intervening optical windows.

The raised profile enhances electrical contact with skin during rolling movement by maintaining engagement over uneven skin contours.

In an embodiment in which the housing 100 is configured as a jar cap, the housing includes a threaded or snap-fit interface configured to attach to a container, such as a skincare product jar. The rolling ball 110 is mounted within the cap such that, when inverted and applied to the skin, the rolling ball protrudes through an opening for skin contact.

The circuit board 120 and battery 126 are disposed within the cap structure, and a sealing element is provided to prevent ingress of product into the electronic components. This configuration enables a user to apply a topical product and subsequently massage the product into the skin using the rolling ball while delivering electrical stimulation and phototherapy.

In an embodiment in which the housing is configured as a mat, the housing comprises a planar, flexible substrate having a plurality of rolling balls 110 mounted therein. The rolling balls are arranged in a grid or array pattern to provide coverage of larger body regions. Each rolling ball is rotatably mounted within a socket formed in the substrate, with electrical connections routed to a central circuit board or distributed control electronics.

The mat includes a power cord for connection to an external power source or a rechargeable battery pack. This configuration permits hands-free treatment, wherein the user rests against the mat, and the rolling balls provide massage, electrical stimulation, and phototherapy to contact regions.

In an embodiment in which the housing is configured as a massager, the rolling ball 110 is integrated into a handheld or wearable massage device. The device further includes vibration motors or additional massage mechanisms. An ergonomic handle or strap facilitates positioning against targeted areas.

The combination of mechanical massage, rolling ball electrotherapy, and phototherapy provides synergistic therapeutic effects compared to a single modality.

In an embodiment, the electrical connection between the circuit board 120 and the electrical stimulation elements accommodates rotation of the rolling ball 110. The conductive shaft 122 or other electrical coupling mechanism transfers power and stimulation signals from the circuit board 120 to the electrodes while permitting free rotation of the ball. Electrical continuity is maintained throughout rotation, enabling uninterrupted delivery of stimulation regardless of the angular position of the rolling ball 110.

In an embodiment, electrical coupling between the patterned conductive traces on the ball surface and the conductive shaft 122 is established via a slip ring, brush contact, conductive bushing, or conductive bearing assembly, as previously described. Conductive rings disposed on an inner surface of the ball adjacent to the mounting axis interface with stationary brush contacts, and internal conductors electrically connect the conductive rings to the patterned conductive traces.

In an embodiment, the conductive shaft 122 includes an externally threaded portion, and a locking member comprising a lock nut having a corresponding internal thread is engaged therewith. The threaded engagement secures the rolling ball 110 onto the shaft, thereby enhancing mechanical stability and preventing displacement during rotation.

In an embodiment, the rear shell includes a support seat configured to receive and secure the battery within the housing cavity. The support seat includes a plurality of heat dissipation slots configured to facilitate thermal management by permitting heat generated by the battery to dissipate from the device. The heat dissipation slots are arranged adjacent to or surrounding the support seat to provide ventilation and maintain safe operating temperatures during therapeutic use.

In an embodiment, the front connector and the rear connector are magnetically coupled. The front connector comprises a magnet, and the rear connector comprises a corresponding magnet or a ferromagnetic element, such as an iron plate. Alternatively, the front connector comprises a ferromagnetic element, and the rear connector comprises a magnet. The magnetic coupling permits tool-free assembly and disassembly of the front shell and rear shell while maintaining secure attachment during therapeutic operation.

In an embodiment, a plurality of massage spheres and a plurality of rolling balls are arranged in two groups. Two sets of massage spheres and two sets of rolling balls are installed on opposite sides of a control switch within the housing. The massage spheres and rolling balls disposed on a first side have a first size, and the massage spheres and rolling balls disposed on a second side have a second size. In certain implementations, the massage spheres on the first side and the massage spheres on the second side differ in size. Alternatively, the massage spheres and rolling balls positioned on the same side have different sizes. The use of varying sizes improves conformity to skin contours and enhances therapeutic effectiveness.

In an embodiment in which the rolling ball is mounted using a floating structure including a spring, the spring is formed of a conductive material. The conductive spring establishes electrical communication with the circuit board, thereby electrically coupling the electrical stimulation element disposed on the rolling ball to the circuit board while permitting limited axial movement of the rolling ball.

When the ball is subjected to an external force, the spring elastically compresses or extends, allowing the ball to move relative to the housing. This floating movement increases and stabilizes contact between the rolling ball and the skin, enabling adaptation to various body contours, including facial, mandibular, and cervical regions.

In an embodiment, the phototherapy unit 118 operates in coordination with the rotation of the rolling ball 110 to deliver light therapy through the light-transmissive region 116. The phototherapy unit 118 emits light toward the transparent rolling ball 110, and the light passes through light-transmissive regions disposed between the electrical stimulation elements. As the ball rotates, different light-transmissive regions align with the phototherapy unit 118, permitting light to pass through the ball and irradiate the skin surface. Continuous light emission combined with rotational movement of the rolling ball provides distributed phototherapy across the treatment area during rolling operation.

The coordinated operation of the electrical stimulation elements and the phototherapy unit through the rotating transparent ball enables dual-modality therapeutic treatment during a single treatment session. Skin tissue receives electrical stimulation through electrodes that contact the skin during rotation of the ball, while simultaneously receiving light therapy through the light-transmissive regions of the transparent rolling ball. The concurrent delivery of electrotherapy and phototherapy provides complementary therapeutic effects as the transparent rolling ball rolls along the skin surface.

1. A therapeutic device, comprising:
a housing having a cavity;
a ball rotatably mounted in the cavity, wherein the ball comprises a transparent material and has a conductive region and a light transmission region, the conductive region having at least one electrical stimulation element disposed thereon;
a phototherapy unit configured to emit light through the light transmission region of the ball; and
a circuit board disposed within the cavity and electrically connected to the at least one electrical stimulation element and the phototherapy unit.

2. The therapeutic device of claim 1, wherein the transparent material comprises at least one of glass, quartz, ceramic, or plastic.

3. The therapeutic device of claim 1, wherein the at least one electrical stimulation element comprises a patterned conductive trace disposed on a surface of the ball, wherein the patterned conductive trace comprises a metal coating applied to the surface of the ball.

4. The therapeutic device of claim 1, wherein the phototherapy unit is positioned external to the ball and within the cavity,.

5. The therapeutic device of claim 1, wherein the at least one electrical stimulation element is configured to provide at least one of EMS therapy, microcurrent therapy, galvanic therapy, or TENS therapy.

6. The therapeutic device of claim 1, wherein the phototherapy unit comprises at least one LED light source.

7. The therapeutic device of claim 1, further comprising a conductive shaft disposed within the cavity, wherein the ball is rotatably mounted on the conductive shaft, and wherein the at least one electrical stimulation element is electrically connected to the circuit board through the conductive shaft.

8. The therapeutic device of claim 1, further comprising a control circuit configured to selectively activate only those of the at least one electrical stimulation element and the phototherapy unit that are in contact with skin during use.

9. The therapeutic device of claim 8, wherein the control circuit is further configured to periodically reverse a polarity of electrical stimulation provided by the at least one electrical stimulation element.

10. The therapeutic device of claim 1, comprising at least two balls rotatably mounted in the cavity.

11. A therapeutic device, comprising:
a transparent ball having a plurality of light emitting diodes integrated therein;
a plurality of patterned conductive traces disposed on an outer surface of the ball, the patterned conductive traces configured to provide electrical stimulation therapy to skin while permitting light from the plurality of light emitting diodes to pass through optical windows defined between the patterned conductive traces; and
a housing supporting the ball, wherein the ball is configured to roll along skin while simultaneously applying electrical stimulation and light therapy.

12. The therapeutic device of claim 11, wherein the patterned conductive traces comprise a metal coating electroplated onto the outer surface of the ball.

13. The therapeutic device of claim 11, wherein the patterned conductive traces comprise raised electrode islands disposed on the outer surface of the ball.

14. The therapeutic device of claim 11, further comprising a control circuit configured to selectively activate only those of the plurality of light emitting diodes and the patterned conductive traces that are in contact with skin during use.

15. The therapeutic device of claim 14, wherein the control circuit is further configured to periodically reverse a polarity of electrical stimulation provided by the patterned conductive traces.

16. The therapeutic device of claim 11, wherein the housing is configured as at least one of a handheld device, a jar cap, a mat, or a massager.

17. A method of providing therapeutic treatment to skin, comprising:
rolling a ball along the skin, the ball comprising a transparent ball having at least one electrical stimulation element disposed on a surface thereof;
delivering electrical stimulation therapy to the skin through the at least one electrical stimulation element while the ball rolls along the skin; and
simultaneously delivering light therapy to the skin through a light transmission area of the transparent ball.

18. The method of claim 17, wherein the at least one electrical stimulation element comprises a patterned conductive trace formed by electroplating a metal coating onto the surface of the transparent ball.

19. The method of claim 17, further comprising selectively activating only those of the at least one electrical stimulation element that are in contact with the skin during use.

20. The method of claim 17, further comprising periodically reversing a polarity of the electrical stimulation therapy.
